(19) United States
(12) Patent Application Publication
(10) Pub. No.: US 2018/0193522 A1
(43) Pub. Date: Jul. 12, 2018

(54) INJECTABLE HYDROGELS AND APPLICATIONS THEREOF

(71) Applicant: LifeCell Corporation, Branchburg, NJ (US)

(72) Inventors: Ming F. Pomerleau, Califon, NJ (US); Hui Xu, Plainsboro, NJ (US)

(21) Appl. No.: 15/846,804

(22) Filed: Dec. 19, 2017

Related U.S. Application Data
(60) Provisional application No. 62/438,599, filed on Dec. 23, 2016.

(51) Int. Cl. A61L 27/20; A61L 27/36; A61L 27/22; A61L 27/52; A61L 27/56

(52) U.S. Cl. CPC A61L 27/20; A61L 27/362; A61L 27/227; A61L 2430/34; A61L 27/52; A61L 27/56; A61L 2400/06; C12Y 111/01007

(57) ABSTRACT

Crosslinked compositions useful for repairing, regenerating, and/or augmenting tissue, as well as acting as a biological scaffold that promotes cell in-growth and tissue integration, are disclosed, as are quick-setting, injectable precursors of such crosslinked compositions. Such crosslinked compositions generally comprise (1) a crosslinked tyramine-substituted hyaluronic acid and (2) an acellular tissue matrix. Also disclosed are methods of repairing, regenerating, and/or augmenting tissues using such crosslinked compositions, particularly voids in human tissue such as anal fistulae.

This application claims priority under 35 U.S.C. § 119 to U.S. Provisional Application No. 62/438,599, which was filed on Dec. 23, 2016, and is herein incorporated by reference in its entirety.

The present disclosure relates generally to crosslinked or cross-linkable compositions that can be used to treat, regenerate, and/or augment tissue. The present disclosure also relates to methods of treating and/or augmenting tissues using such compositions.

Treatment of voids or other defects in both hard and soft tissue can present challenges due to their often irregular or even unknown geometries, such as in the repair of complex anal fistulae. Filler materials can be used for the treatment of such tissue and can conform to and set into the irregular or unknown geometries of such voids in vivo. These filler materials, however, should be able to resist migration, retain their volume and structural integrity over time, integrate well with surrounding tissue in a short amount of time, and/or promote cell in-growth and tissue regeneration.

Semi-permanent and permanent injectable filler materials currently approved as aesthetic dermal fillers have been contemplated for use in the treatment of hard and soft tissue voids, particularly for the treatment of complex anal fistulae. However, many dermal fillers are responsible for both short- and long-term clinical complications that are product-treatment related. See de Vries, et al., Expert Review of Medical Devices, Vol. 10(6), pp. 835-53 (2013). For example, synthetic materials, such as cyanoacrylate glue, and biologically derived materials, such as BIOGLUE®, have been studied as biological infill materials in the treatment of anal fistulae. Lewis, et al., Colorectal Disease, Vol. 14, pp 1445-56 (2012). However, with regard to cyanoacrylate glue, histological data has shown that it acts as a barrier to host tissue integration, initiates a chronic inflammatory response, and can cause multiple abscess formation. Id. at 1447. Thus, the concern is that if used as an infill material, the glue will act in a palliative fashion by completely occluding the fistula until it starts degrading, resulting in either a recurrent fistula or acute sepsis. Id. Meanwhile, BIOGLUE® is associated with unacceptable rates of acute sepsis, often requiring surgical drainage, and may cause nerve injury, coagulation necrosis, and release glutaraldehyde levels that are toxic. Id. at 1448.

Cross-linked hyaluronic acid (HA) has become a desirable material for use in soft tissue augmentation. However, HA lacks the long term structural integrity and tissue integration and regenerative ability necessary for fistula repair. Accordingly, there exists a continued need for improved injectable filler materials that retain their volume and structural integrity over time, integrate well with surrounding tissue, promote cell in-growth, and are clinically safe.

The present disclosure provides for injectable filler materials that provide one or more of the aforementioned properties, as well as for methods of their use.

Thus, according to various embodiments, a composition comprising (1) a tyramine-substituted hyaluronic acid and (2) an acellular tissue matrix is provided.

In certain embodiments, the tyramine-substituted hyaluronic acid of the above composition is derived from a hyaluronic acid having a molecular weight of up to 10 MDa. In certain embodiments, the tyramine-substituted hyaluronic acid of the above composition is derived from a hyaluronic acid selected from a group consisting of human-derived hyaluronic acid, porcine-derived hyaluronic acid, bovine-derived hyaluronic acid, bacteria recombinant hyaluronic acid, rooster comb hyaluronic acid, or any combination thereof. In certain embodiments, the tyramine-substituted hyaluronic acid is present in the above composition in a concentration of up to 25 mg/mL, based on the total volume of the composition.

In certain embodiments, the acellular tissue matrix of the above composition is derived from dermal tissue, adipose tissue, muscle tissue, bone tissue, cartilage tissue, or any combination thereof. In certain embodiments, the acellular tissue matrix used to form the above composition is in the form of a wet slurry, diced tissue particles, a cryomilled dry powder, micronized dry particles, or freeze dried porous sponge particles.

In certain embodiments, the dry weight ratio of tyramine-substituted hyaluronic acid to acellular tissue matrix in the above composition is in the range of from 1.0:1.0 to 1.0:100.0.

In certain embodiments, the above composition further comprises a peroxidase. In certain of those embodiments, the peroxidase is horseradish peroxidase. In certain of those embodiments, the units of activity per volume of the horseradish peroxidase in the above composition is in the range of from 0.5 U/mL to 50 U/mL, based on the total volume of the composition.

In certain embodiments, the above composition is in the form of a liquid. In certain of those embodiments, the above composition is in the form of a solution, a suspension, a dispersion, or any combination thereof. In certain of those embodiments, the above composition comprises water. In certain of those embodiments, the above composition comprises an aqueous buffer solution.

In certain embodiments, the acellular tissue matrix of the above composition has been sterilized. In certain embodiments, the acellular tissue matrix has been sterilized via e-beam, gamma radiation, UV radiation, and/or supercritical CO₂.

According to other embodiments, a crosslinked composition prepared by mixing the above composition further comprising a peroxidase with hydrogen peroxide is provided. In certain embodiments, the above crosslinked composition is prepared by mixing the above composition further comprising a peroxidase with an aqueous solution of hydrogen peroxide having a hydrogen peroxide concentration in the range of from 0.001 to 0.1% by weight. In certain of those embodiments, the above crosslinked composition is prepared by mixing the above composition further comprising a peroxidase with a volume of aqueous solution of hydrogen peroxide in the range of from 40 μL to 1200 μL for every 1 mL of tyramine-substituted hyaluronic acid having a concentration of 25 mg/mL. In certain embodiments, the above crosslinked composition is in the form of a hydrogel.

According to other embodiments, a composition comprising (1) a crosslinked or cross-linkable tyramine-substituted hyaluronic acid and (2) an acellular tissue matrix is provided. In certain embodiments, the crosslinked tyramine-substituted hyaluronic acid of the above composition comprises a crosslinking structure of formula (I):

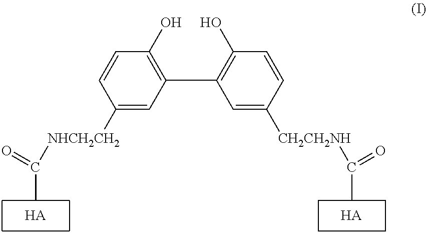

wherein each HA is the same or a different crosslinked tyramine-substituted hyaluronic acid. In certain embodiments, the above composition is in the form of a hydrogel. In certain embodiments, the above composition comprises a buffered aqueous solution. In certain embodiments, the crosslinked or crosslinkable tyramine-substituted hyaluronic acid of the above composition is derived from a hyaluronic acid having a molecular weight in the range of about 1.5 MDa to about 1.8 MDa. In certain embodiments, the tyramine-substituted hyaluronic acid is of the above composition is derived from a hyaluronic acid selected from a group consisting of human-derived hyaluronic acid, porcine-derived hyaluronic acid, bovine-derived hyaluronic acid, or any combination thereof. In certain embodiments, the acellular tissue matrix of the above composition is derived from dermal tissue, adipose tissue, muscle tissue, bone tissue, cartilage tissue or any combination thereof. In certain embodiments, the acellular tissue matrix of the above composition has been sterilized. In certain embodiments, the acellular tissue matrix has been sterilized via e-beam, gamma radiation, UV radiation, and/or supercritical CO₂.

According to other embodiments, a method of treating and/or augmenting tissue in a human or an animal is provided. The method can comprise the steps of (a) providing an aqueous composition comprising (1) a tyramine-substituted hyaluronic acid, (2) an acellular tissue matrix, and (3) a peroxidase, (b) providing an aqueous solution of hydrogen peroxide, (c) mixing the aqueous composition of (a) and the aqueous solution of (b) to form a mixture and initiate crosslinking of the tyramine-substituted hyaluronic acid, (d) introducing the mixture of (c) into the tissue of a person or animal to be treated and/or augmented such that the crosslinking of the tyramine-substituted hyaluronic acid is completed in situ. In certain embodiments, the peroxidase used in the above method is horseradish peroxidase. In certain embodiments, the tyramine-substituted hyaluronic acid used in the above method is derived from a hyaluronic acid selected from a group consisting of human-derived hyaluronic acid, porcine-derived hyaluronic acid, bovine-derived hyaluronic acid, bacteria recombinant hyaluronic acid, rooster comb hyaluronic acid, or any combination thereof. In certain embodiments, the acellular tissue matrix used in the above method is derived from dermal tissue, adipose tissue, muscle tissue, bone tissue, cartilage tissue, or any combination thereof. In certain embodiments, the acellular tissue matrix used in the above method has been sterilized prior to step (a). In certain embodiments, the acellular tissue matrix has been sterilized via e-beam, gamma radiation, UV radiation, and/or supercritical CO₂.

According to other embodiments, a method of filling a void in the tissue of a human or an animal comprising the steps of (a) providing an aqueous composition comprising (1) a tyramine-substituted hyaluronic acid, (2) an acellular tissue matrix, and (3) a peroxidase, (b) providing an aqueous solution of hydrogen peroxide, (c) mixing the aqueous composition of (a) and the aqueous solution of (b) to form a mixture and initiate crosslinking of the tyramine-substituted hyaluronic acid, (d) introducing the mixture of (c) into the void in the tissue of the person or animal to be filled such that the void is filled and the crosslinking of the tyramine-substituted hyaluronic acid is completed in situ. In certain embodiments, the peroxidase used in the above method is horseradish peroxidase. In certain embodiments, the acellular tissue matrix is derived from dermal tissue. In certain embodiments, the void is an anal fistula in a human. In certain embodiments, the acellular tissue matrix used in the above method has been sterilized prior to step (a). In certain embodiments, the acellular tissue matrix has been sterilized via e-beam, gamma radiation, UV radiation, and/or supercritical CO₂.

According to other embodiments, a kit comprising (1) a tyramine-substituted hyaluronic acid and (2) an acellular tissue matrix is provided. In certain embodiments, the kit further comprises (3) a peroxidase. In certain embodiments, the kit further comprises (4) a peroxide. In certain embodiments, the kit further comprises a device capable of mixing components (1), (2), (3), and (4) and/or injecting a mixture of components (1), (2), (3), and (4). In certain embodiments, the device is selected from a group consisting of single barrel syringes, dual barrel syringe systems, cannulae, syringe-to-syringe luer lock adapter-based systems, in-line static mixers, mixing tips, or any combination thereof.

### BRIEF DESCRIPTION OF THE DRAWINGS

The foregoing and other features and advantages provided by the present disclosure will be more fully understood from the following description of exemplary embodiments when read together with the accompanying drawings.

FIG. 1a is a photograph of a haematoxylin and eosin (i.e., H&E) stained section at 4× magnification of a rat explant with little inflammation after a 4-week subcutaneous exposure to a puck of the crosslinked hydrogel of Example 1.

FIG. 1b is a photograph of the gross rat explant from FIG. 1a.

FIG. 2a is a photograph of an haematoxylin and eosin (i.e., H&E) stained section at 4× magnification of a rat explant with little inflammation after a 4-week subcutaneous exposure to a puck of the crosslinked hydrogel of Example 1.

FIG. 2b is a photograph of the gross rat explant from FIG. 2a.

FIG. 3a is a photograph of an haematoxylin and eosin (i.e., H&E) stained section at 4× magnification of a rat explant with little inflammation after a 4-week subcutaneous exposure to a puck of the crosslinked hydrogel of Example 1.

FIG. 3b is a photograph of the gross rat explant from FIG. 3a.

FIG. 4 is a photograph of an haematoxylin and eosin (i.e., H&E) stained section at 10× magnification of a rat explant with moderate cell infiltration after a 4-week subcutaneous exposure to a puck of the crosslinked hydrogel of Example 1.

FIG. 5 is a photograph of an haematoxylin and eosin (i.e., H&E) stained section at 10× magnification of a rat explant with moderate cell infiltration after a 4-week subcutaneous exposure to a puck of the crosslinked hydrogel of Example 1.

FIG. 6 is a photograph of an haematoxylin and eosin (i.e., H&E) stained section at 10× magnification of a rat explant with moderate cell infiltration after a 4-week subcutaneous exposure to a puck of the crosslinked hydrogel of Example 1.

FIG. 7 is a photograph of an haematoxylin and eosin (i.e., H&E) stained section at 20× magnification of a rat explant with good cell infiltration after a 4-week subcutaneous exposure to an implant of acellular dermal matrix slurry (ADMS).

FIG. 8 is a photograph of an haematoxylin and eosin (i.e., H&E) stained section at 20× magnification of a rat explant with good cell infiltration after a 4-week subcutaneous exposure to the 5:1 ADMS:tyrHA ratio crosslinked hydrogel implant of Example 3.

FIG. 9 is a photograph of an haematoxylin and eosin (i.e., H&E) stained section at 20× magnification of a rat explant with good cell infiltration after a 4-week subcutaneous exposure to the 12:1 ADMS:tyrHA ratio crosslinked hydrogel implant of Example 3.

### DESCRIPTION OF EXEMPLARY EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Any ranges described herein will be understood to include the endpoints and all values between the endpoints.

Any section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

The present disclosure provides for tissue filler materials. The materials can be injectable, quick-crosslinking filler materials for use in the treatment and/or augmentation of voids in both hard and soft tissue. The materials can act as biological scaffolds that conform to irregular and/or unknown three-dimensional geometries in vivo, stay in the desired location, retain their volume and structural integrity over time, integrate well with surrounding tissue, and/or promote cell in-growth and regeneration. Prior to crosslinking, these disclosed filler materials are injectable precursor compositions that, at a minimum, comprise (1) a tyramine-substituted hyaluronic acid and (2) an acellular tissue matrix.

The tyramine-substituted hyaluronic acid (tyrHA) of the presently disclosed precursor compositions is derived from hyaluronic acid (HA), otherwise known as hyaluronan, that has been chemically modified to contain hydroxyphenyl groups. HA is common in biologic materials and is concentrated in specialized tissues such as cartilage, vocal cords, vitreous, synovial fluid, umbilical cord, and dermis. In these tissues, its function is manifold, influencing tissue viscosity, shock absorption, wound healing, and space filling. HA has been shown to influence many processes within the extracellular matrix (ECM) in native tissues including matrix assembly, cell proliferation, cell migration, and embryonic/tissue development.

HA is composed of repeating pairs of glucuronic acid (glcA) and N-acetylglucosamine (glcNAc) residues linked by a β-1,3-glycosidic bond, as shown in the following structure:

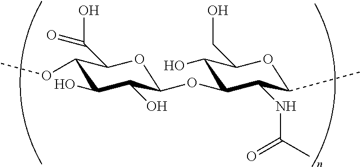

For each HA chain, this simple disaccharide is repeated up to 10,000 times or greater resulting in macromolecules that can have a molecular weight of up to 10 million daltons (i.e., 10 MDa). Therefore, in certain embodiments, the tyrHA of the presently disclosed precursor compositions is derived from an HA having a molecular weight of up to about 1 kDa, about 2 kDa, about 3 kDa, about 4 kDa, about 5 kDa, about 6 kDa, about 7 kDa, about 8 kDa, about 9 kDa, about 10 kDa, about 20 kDa, about 30 kDa, about 40 kDa, about 50 kDa, about 60 kDa, about 70 kDa, about 80 kDa, about 90 kDa, about 100 kDa, about 200 kDa, about 300 kDa, about 400 kDa, about 500 kDa, about 600 kDa, about 700 kDa, about 800 kDa, about 900 kDa, about 1 MDa, about 2 MDa, about 3 MDa, about 4 MDa, about 5 MDa, about 6 MDa, about 7 MDa, about 8 MDa, about 9 MDa, and about 10 MDa. In certain embodiments, the tyrHA of the presently disclosed precursor compositions is derived from an HA having a molecular weight in the range of from about 1 MDa to about 2 MDa. In certain embodiments, the tyrHA of the presently disclosed precursor compositions is derived from an HA having a molecular weight in the range of from about 1.5 MDa to about 1.8 MDa.

Adjacent disaccharide units of HA are linked by a β-1,4-glycosidic bond. Each glcA residue has a carboxylic acid group (CO₂H) attached to the number 5 carbon atom of the glucose ring. Under biological conditions, HA is a negatively charged, randomly coiled polymer filling a volume more than 1,000 times greater than would be expected based on molecular weight and composition alone. As noted above, the strong negative charges attract cations and water, which allow HA to assume the form of a strongly hydrated gel in vivo, giving it a unique viscoelastic and shock-absorbing property. HA represents a readily available and desirable scaffolding material for tissue engineering applications as it is non-immunogenic, non-toxic, and non-inflammatory. Also, as a naturally occurring extracellular matrix (ECM) molecule, it offers the advantages of being recognized by cell receptors, of interacting with other ECM molecules, and/or of being metabolized by normal physiological pathways.

In certain embodiments, the tyrHA of the presently disclosed precursor compositions is derived from an HA selected from a group consisting of human-derived HA, porcine-derived HA, bovine-derived HA, bacteria recombinant hyaluronic acid, rooster comb hyaluronic acid or any combination thereof.

The glucuronic acid residue provides a carboxyl group periodically along the repeat disaccharide structure of HA that is available for hydroxyphenyl group substitution. The hydroxyphenyl group is introduced by reaction of HA with tyramine. Tyramine is a phenolic molecule having an ethyl amine group attached para to the OH group on the benzene ring, as depicted in the following structure:

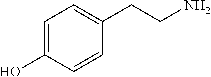

The mechanism for tyramine substitution onto the singly bound oxygen atom of a CO₂H group on HA can proceed via a carbodiimide-mediated reaction mechanism, as illustrated below in Scheme 1:

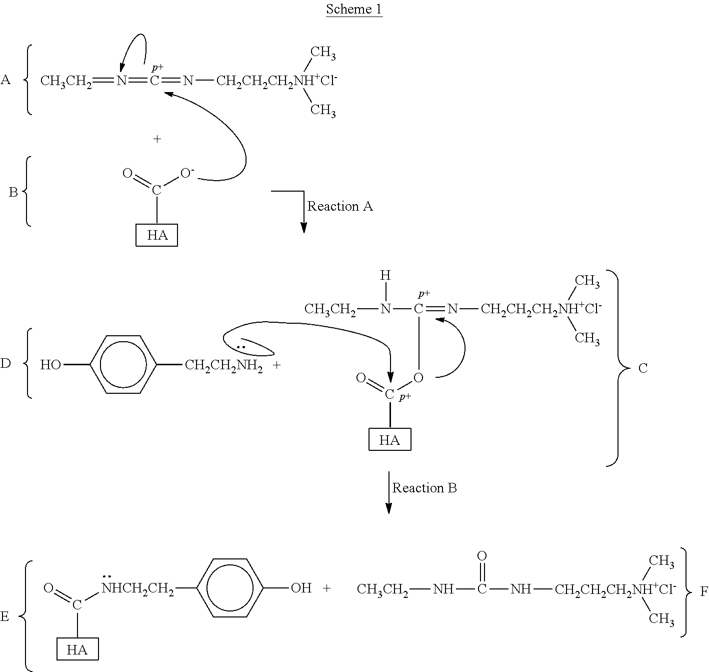

wherein structure A is the carbodiimide, 1-ethyl-3-(3-dimethylaminopropyl)carbodiimide (EDC), structure B is HA (though only one CO₂H group is shown), structure C is the product of Reaction A and is 1-ethyl-3-(3-dimethylaminopropyl) isourea, structure D is tyramine, structure E is tyrHA, and structure F is 1-ethyl-3-(3-dimethylaminopropyl)urea (EDU).

In the above reaction pathway, a negatively charged oxygen atom of the carboxyl group of the HA molecule attacks, via a nucleophilic reaction mechanism, the electron-deficient diamide carbon atom on the carbodiimide molecule (EDC) to form the activated O-acylisourea (Reaction A). The result is that the carbon atom of the HA carboxylate group becomes sufficiently electron deficient to be susceptible to nucleophilic attack by the unshared pair of electrons on the amine group of a tyramine molecule (Reaction B). Reaction A is preferably catalyzed by a suitable catalyst that will result in the formation of an active ester during Reaction A, thus permitting the reaction to be carried out at substantially neutral pH (e.g., pH 6.5). Examples of suitable catalysts include, but are not limited to, N-hydroxysuccinimide (NHS), 1-hydroxybenzotriazole (HOBt), and N-hydroxysulfosuccinimide (NHSS). Examples of other suitable carbodiimides besides EDC include, but are not limited to, 1-cyclohexyl-3-[2-(4-methylmorpholino)ethyl]carbodiimide (CMC) and dicyclohexylcarbodiimide (DCC).

The result of Reaction A above is O-acylisourea-substituted HA. Essentially, the EDC molecule has been temporarily substituted onto the carboxylic acid group of a glcA residue of the HA molecule, making the carbon atom of the carboxylic acid group slightly positively charged. The electron pair from the terminal amine group of a tyramine molecule is then substituted onto the carbon atom via a nucleophilic substitution reaction as explained in the preceding paragraph (Reaction B). The result of Reaction B is the tyrHA molecule and acylurea, a byproduct. It will be understood by persons of ordinary skill in the art that Reactions A and B will result in a plurality of tyramine substitutions on the periodic glcA residues of HA molecules; a single substitution has been shown here for brevity and clarity.

As used herein, the term “acellular tissue matrix” refers generally to any tissue matrix that is substantially free of native cells and/or native cellular components. The acellular tissue matrices of the presently disclosed precursor compositions may be derived from any type of tissue. Examples of the tissues that may be used to construct the acellular tissue matrices of the presently disclosed precursor compositions include, but are not limited to, skin (i.e., dermal), parts of skin (e.g., dermis), adipose, fascia, muscle (striated, smooth, or cardiac), pericardial tissue, dura, umbilical cord tissue, placental tissue, cardiac valve tissue, ligament tissue, tendon tissue, blood vessel tissue, such as arterial and venous tissue, cartilage, bone, neural connective tissue, urinary bladder tissue, ureter tissue, and intestinal tissue.

The acellular tissue matrices of the presently disclosed precursor compositions can be selected to provide a variety of different biological and/or mechanical properties. For example, an acellular tissue matrix can be selected to allow tissue in-growth and remodeling to assist in regeneration of tissue normally found at the site where the matrix is implanted. In certain embodiments, the acellular tissue matrices of the present disclosure can be selected from ALLODERM® or STRATTICE™ (LIFECELL CORPORATION, Branchburg, N.J.), which are human and porcine acellular dermal matrices, respectively. In certain other embodiments, the particulate acellular tissue matrix can include CYMETRA®, LifeCell Corporation, Branchburg, N.J., which is a particulate acellular dermal matrix. In certain other embodiments, the acellular tissue matrix can include demineralized bone matrix (i.e., DBM). Alternatively, other suitable acellular tissue matrices can be used, as described further below.

Tissue matrices can be processed in a variety of ways to produce decellularized (i.e., acellular) tissue matrices. In general, the steps involved in the production of an acellular tissue matrix include harvesting the tissue from a donor (e.g., a human cadaver or animal source) and cell removal under conditions that preserve biological and structural function. In certain embodiments, the process includes chemical treatment to stabilize the tissue and avoid biochemical and structural degradation together with or before cell removal. In various embodiments, the stabilizing solution arrests and prevents osmotic, hypoxic, autolytic, and proteolytic degradation, protects against microbial contamination, and reduces mechanical damage that can occur with tissues that contain, for example, smooth muscle components (e.g., blood vessels). The stabilizing solution may contain an appropriate buffer, one or more antioxidants, one or more oncotic agents, one or more antibiotics, one or more protease inhibitors, and/or one or more smooth muscle relaxants.

The tissue is then placed in a decellularization solution to remove viable cells (e.g., epithelial cells, endothelial cells, smooth muscle cells, and fibroblasts) from the structural matrix without damaging its biological and structural integrity. The decellularization solution may contain an appropriate buffer, salt, an antibiotic, one or more detergents, one or more agents to prevent crosslinking, one or more protease inhibitors, and/or one or more enzymes.

Acellular tissue matrices can be tested or evaluated to determine if they are substantially free of cell and/or cellular components in a number of ways. For example, processed tissues can be inspected with light microscopy to determine if cells (live or dead) and/or cellular components remain. In addition, certain assays can be used to identify the presence of cells or cellular components. For example, DNA or other nucleic acid assays can be used to quantify remaining nuclear materials within the tissue matrices. Generally, the absence of remaining DNA or other nucleic acids will be indicative of complete decellularization (i.e., removal of cells and/or cellular components). Finally, other assays, e.g., immunohistochemical stainings, that identify cell-specific components (e.g., surface antigens) can be used to determine if the tissue matrices are acellular. After the decellularization process, the tissue sample is washed thoroughly with saline.

While an acellular tissue matrix may be made from one or more individuals of the same species as the recipient of the acellular tissue matrix, this need not necessarily be the case. Thus, for example, an acellular tissue matrix may be made from porcine tissue and implanted in a human patient. Species that can serve as recipients of acellular tissue matrix and donors of tissues or organs for the production of the acellular tissue matrix include, without limitation, mammals, such as humans, nonhuman primates (e.g., monkeys, baboons, or chimpanzees), pigs, cows, horses, goats, sheep, dogs, cats, rabbits, guinea pigs, gerbils, hamsters, rats, or mice.

Elimination of the α-gal epitopes from the collagen-containing material may diminish the immune response against the collagen-containing material. The α-gal epitope is expressed in non-primate mammals and in New World monkeys (monkeys of South America) as well as on macromolecules such as proteoglycans of the extracellular components. U. Galili et al., J. Biol. Chem., 263: 17755 (1988). This epitope is absent in Old World primates (monkeys of Asia and Africa and apes) and humans, however. Id. Anti-gal antibodies are produced in humans and primates as a result of an immune response to α-gal epitope carbohydrate structures on gastrointestinal bacteria. U. Galili et al., Infect. Immun., 56:1730 (1988); R. M. Hamadeh et al., J. Clin. Invest., 89:1223 (1992).

Accordingly, in certain embodiments, when animals that produce α-gal epitopes are used as the tissue source, the substantial elimination of α-gal epitopes from cells and from extracellular components of the acellular tissue matrix, and the prevention of re-expression of cellular α-gal epitopes can diminish the immune response against the acellular tissue matrix associated with anti-gal antibody binding to α-gal epitopes.

To remove α-gal epitopes, the tissue sample may be subjected to one or more enzymatic treatments to remove certain immunogenic antigens, if present in the sample. In some embodiments, the tissue sample may be treated with an α-galactosidase enzyme to eliminate α-gal epitopes if present in the tissue. Any suitable enzyme concentration and buffer can be used as long as sufficient removal of antigens is achieved.

Alternatively, rather than treating the tissue with enzymes, animals that have been genetically modified to lack one or more antigenic epitopes may be selected as the tissue source. For example, animals (e.g., pigs) that have been genetically engineered to lack the terminal α-galactose moiety can be selected as the tissue source. For descriptions of appropriate animals see U.S. Patent Application Pub. No. 2005/0028228 A1 and U.S. Pat. No. 6,166,288, the disclosures of which are incorporated herein by reference in their entirety. In addition, certain exemplary methods of processing tissues to produce acellular tissue matrices with or without reduced amounts of or lacking alpha-1,3-galactose moieties, are described in Xu, Hui et al., “A Porcine-Derived Acellular Dermal Scaffold that Supports Soft Tissue Regeneration: Removal of Terminal Galactose-α-(1,3)-Galactose and Retention of Matrix Structure,” Tissue Engineering, Vol. 15, 1-13 (2009), which is incorporated by reference in its entirety.

In certain embodiments, the acellular tissue matrix can be sterilized prior to use. Sterilization of the acellular tissue matrix can be achieved by any suitable means known in the art. Examples of such means include, but are not limited to, sterilization via e-beam, gamma radiation, UV radiation, and/or supercritical CO₂.

In certain embodiments, the presently disclosed precursor compositions can be formed by thoroughly physically mixing the tyrHA and the acellular tissue matrix components. These components can be mixed by any means known in the art. When they are mixed together, both the tyrHA and the acellular tissue matrix components used to form the presently disclosed precursor compositions can be in any suitable physical form that allows for their mixture with each other and that ultimately does not interfere with the crosslinking of the tyrHA. Examples of such physical forms for the tyrHA include, but are not limited to, solid physical forms, such as a lyophilized powders, and liquid physical forms, such as solutions, suspensions, dispersions, or any combination thereof. In certain embodiments, these solutions, suspensions, or dispersions are aqueous solutions, suspensions, or dispersions. In certain embodiments, the medium for such solutions, suspensions, and dispersions is water or an aqueous buffer solution. Examples of such physical forms for the acellular tissue matrix include, but are not limited to, slurries, diced tissue particles, cryomilled dry powders, micronized dry particles, and freeze dried porous sponge particles. In certain embodiments, the acellular tissue matrix is in the form of a slurry having a solid content in the range of from 10% to 25% by weight and where the liquid medium is an aqueous buffer or a preservation solution.

The tyrHA can be present in the precursor composition in any suitable concentration. In certain embodiments, the tyrHA is present in the precursor composition in a concentration of up to 25 mg/mL, based on the total volume of the composition. Examples of such concentrations include, but are not limited to, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1.0 mg/mL, 2.0 mg/mL, 3.0 mg/mL, 4.0 mg/mL, 5.0 mg/mL, 6.0 mg/mL, 7.0 mg/mL, 8.0 mg/mL, 9.0 mg/mL, 10.0 mg/mL, 11.0 mg/mL, 12.0 mg/mL, 13.0 mg/mL, 14.0 mg/mL, 15.0 mg/mL, 16.0 mg/mL, 17.0 mg/mL, 18.0 mg/mL, 19.0 mg/mL, 20.0 mg/mL, 21.0 mg/mL, 22.0 mg/mL, 23.0 mg/mL, 24.0 mg/mL, and 25.0 mg/mL, based on the total volume of the composition.

The tyrHA and the acellular tissue matrix can be present in the precursor composition in any suitable weight ratio to each other. In certain embodiments, the dry weight ratio of tyrHA and the acellular tissue matrix in the precursor composition is in the range of from 1.0:1.0 to 1.0:100.0. In certain embodiments, the dry weight ratio of tyrHA and the acellular tissue matrix in the precursor composition is in the range of from 1:25 to 1:90. In certain other embodiments, the dry weight ratio of tyrHA and the acellular tissue matrix in the precursor composition is in the range of from 1.0:7.2 to 1.0:36.0. Examples of such concentrations include, but are not limited to, 1.0:7.2, 1.0:8.0, 1.0:9.0, 1.0:10.0, 1.0:11.0, 1.0:12.0, 1.0:13.0, 1.0:14.0, 1.0:15.0, 1.0:16.0, 1.0:17.0, 1.0:18.0, 1.0:19.0, 1.0:20.0, 1.0:21.0, 1.0:22.0, 1.0:23.0, 1.0:24.0, 1.025.0, 1.0:26.0, 1.0:27.0, 1.0:28.0, 1.0:29.0, 1.0:30.0, 1.0:31.0, 1.0:32.0, 1.0:33.0, 1.0:34.0, 1.0:35.0, and 1.0:36.0.

In certain embodiments, the presently disclosed precursor composition further comprises a peroxidase. Examples of such peroxidases include, but are not limited to, horseradish peroxidase, hematin, and soybean peroxidase. The peroxidase can be present in the precursor composition in any suitable concentration. In certain embodiments, the peroxidase is present in the precursor composition in a concentration in the range of from 0.5 U/mL to 50 U/mL, based on the total volume of the composition. Examples of such concentrations include, but are not limited to, 1 U/mL, 1.5 U/mL, 2 U/mL, 2.5 U/mL, 3 U/mL, 3.5 U/mL, 4 U/mL, 4.5 U/mL, 5 U/mL, 5.5 U/mL, 6 U/mL, 6.5 U/mL, 7 U/mL, 7.5 U/mL, 8 U/mL, 8.5 U/mL, 9 U/mL, 9.5 U/mL, and 10 U/mL, based on the total volume of the composition.

The concentrations of tyrHA, peroxidase, hydrogen peroxide, the weight ratios of tyrHA to acellular tissue matrix relative to each other, and the mixing method may, individually or collectively, be varied in order to modulate the curing time of the tyramine-substituted hyaluronic acid.

The presently disclosed precursor compositions can be in any suitable flowable form. In certain embodiments, the presently disclosed precursor compositions can be in any flowable form suitable for injection. In certain embodiments, the precursor composition is in the form of a liquid. In certain embodiments, the precursor compound is in the form of an aqueous liquid. In certain embodiments, the precursor compound is in the form of a solution, a suspension, a dispersion, or any combination thereof. In certain embodiments, the medium for such solutions, suspensions, and dispersions is water or an aqueous buffer solution. Alternatively, the presently disclosed precursor compositions can be in solid form, such as a lyophilized powder, right up until prior to use, when it is then reconstituted to a suitable form for injection (i.e., a solution, suspension, dispersion, or any combination thereof) by addition of water or an aqueous buffer solution to the solid. The presently disclosed precursor compositions can have any viscosity suitable for injection.

In certain embodiments, the viscosity of the presently disclosed precursor compositions can be modulated by modulating the particle size of acellular tissue matrix (i.e., viscosity is increased or decreased as particle size is increased or decreased, respectively). The acellular tissue matrix can be of any particle size suitable for injection. In certain embodiments, the viscosity of the presently disclosed precursor compositions is such that it can pass through a 27 G needle or if desired, needles of smaller diameter. In certain embodiments, the particle size of the acellular tissue matrices of such precursor compositions is 250 microns or less. In certain embodiments, the particle size of the acellular tissue matrices of such precursor compositions is less than 1 micron. In certain embodiments, the particle size of the acellular tissue matrices of such precursor compositions is about 1 μM, 2 μM, 3 μM, 4 μM, 5 μM, 6 μM, 7 μM, 8 μM, 9 μM, 10 μM, 15 μM, 20 μM, 25 μM, 30 μM, 35 μM, 40 μM, 45 μM, 50 μM, 55 μM, 60 μM, 65 μM, 70 μM, 75 μM, 80 μM, 85 μM, 90 μM, 95 μM, 100 μM, 105 μM, 110 μM, 115 μM, 120 μM, 125 μM, 130 μM, 135 μM, 140 μM, 145 μM, 150 μM, 155 μM, 160 μM, 165 μM, 170 μM, 175 μM, 180 μM, 185 μM, 190 μM, 195M, 200 μM, 205 μM, 2101 μM, 2155 μM, 220 μM, 225 μM, 230 μM, 235 μM, 240 μM, 245 μM, or 250 μM. In certain other embodiments, the particle size of the acellular tissue matrices of such precursor compositions is greater than 250 microns.

The presently disclosed precursor compositions can be administered to a human or an animal to repair and/or augment tissue. Because the crosslinking reaction requires both a peroxide and a peroxidase, separate aqueous compositions containing each of these components can be prepared for convenient application to a surgical site. Thus, in certain embodiments, the method of administration comprises providing a first aqueous composition comprising a mixture of (1) a tyrHA and (2) an acellular tissue matrix, along with either a peroxidase or a peroxide, but not both, while separately providing a second aqueous composition comprising the peroxidase or the peroxide not provided in the first aqueous composition. The first and second aqueous compositions are then mixed to initiate crosslinking of the tyramine-substituted hyaluronic acid. This mixture is then introduced into the tissue of the person or animal to be treated and/or augmented and the crosslinking of the tyramine-substituted hyaluronic acid is completed in situ. In certain embodiments, the tissue comprises a void, such as a fistula (e.g., anal fistula) or abdominal wall defect (e.g., hemia, inguinal hemia, or other abdominal wall defect), to be filled and the mixture is then introduced into the void in the tissue of the person or animal such that the void is partially or completely filled and the crosslinking of the tyramine-substituted hyaluronic acid is completed in situ. In certain embodiments, the first aqueous composition comprises a tyrHA, an acellular tissue matrix, and a peroxidase, while the second aqueous composition is an aqueous solution of hydrogen peroxide. In certain other embodiments, the first aqueous composition comprises a tyrHA, an acellular tissue matrix, and hydrogen peroxide, while the second aqueous composition comprises a peroxidase.

Any suitable peroxide may be used in the above method. An example of a suitable peroxide includes, but is not limited to, hydrogen peroxide, benzoyl peroxide, lipid peroxides, and other organic hydroperoxides. Any suitable concentration of peroxide can be used in the above method. In certain embodiments, the peroxide is used in the above method in a concentration in the range of from 0.001 to 1.0% by weight. Examples of such concentrations include, but are not limited to, 0.01%, 0.02%, 0.03%, 0.04%, 0.05%, 0.06%, 0.07%, 0.08%, 0.09%, 0.10%, 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, and 1.0% by weight. In certain embodiments, the peroxide is used in the above method in a concentration in the range of from 0.001 to 0.03% by weight. In certain embodiments, the volume of peroxide used in the above method is in the range of from 10 μL to 1200 μL for every 1 mL of tyramine-substituted hyaluronic acid having a concentration of 25 mg/mL. In certain embodiments, the volume of hydrogen peroxide used in the above method is 1200 uL of a 0.001% by weight aqueous solution, 120 μL of a 0.01% by weight aqueous solution, 240 μL of a 0.01% by weight aqueous solution, 40 μL of a 0.03% by weight aqueous solution, or 120 μL of a 0.03% by weight aqueous solution for every 1 mL of tyramine-substituted hyaluronic acid having a concentration of 25 mg/mL. In certain embodiments, the concentration of the peroxide used in the above method can be diluted with water so to increase the water content in the presently disclosed precursor compositions, which can, in turn, enhance its flowability.

The first aqueous composition comprising the precursor composition and either the peroxidase or peroxide, as well as the second aqueous composition comprising the peroxidase or peroxide not provided in the first aqueous composition can be administered to a human or an animal by any suitable means known in the art. Examples of such means include, but are not limited to, single barrel syringes, dual barrel syringe systems, and cannulae. In certain embodiments, both the first and second aqueous compositions can be first mixed to initiate crosslinking and then the mixture can be subsequently introduced into the tissue of the human or animal to be treated and/or augmented. In certain of those embodiments, the first and second aqueous compositions can be mixed by any suitable means known in the art. Examples of such suitable means include, but are not limited to, syringe-to-syringe luer lock adapter-based systems and in-line static mixers and mixing tips. In certain embodiments, both the first and second aqueous compositions can be simultaneously introduced into the tissue of the human or animal to be treated and/or augmented and mixed to initiate crosslinking. In certain embodiments, the material can be injected into the face using a 27 gauge or smaller needle. In certain embodiments, the material can be delivered to anal fistulae using a cannula.

In certain embodiments, the above method of treating and/or augmenting tissue in a human or an animal involves filling a void in the tissue of a human or an animal. In certain embodiments, the void in the tissue is the result of damage or loss of tissue due to various diseases and/or structural damage (e.g., from trauma, surgery, atrophy, and/or long-term wear and degeneration). Examples of such voids include, but are not limited to, simple and complex anal fistulae, osteochondral defects (i.e., defects in bone and/or cartilage), tunneling wounds, hernias (e.g., inguinal hernias), and other deep wounds to both soft (e.g., muscle) and hard (e.g., bone) tissue. Furthermore, the presently disclosed precursor compounds, as well as the resulting crosslinked hydrogels, can also be used to aesthetically (i.e., cosmetically) augment tissue. Thus, in certain other embodiments, the precursor composition can be injected into the tissue of a human and crosslinked to create an aesthetic tissue augmentation implant. Examples of human tissues that can be aesthetically augmented using the presently disclosed compositions include, but are not limited to, breast tissue, buttock tissue, chest tissue, thigh tissue, calf tissue, and facial tissue, including lip, nasolabial folds, and cheek tissue. Examples of particular cosmetic applications for which the presently disclosed precursor compounds, as well as the resulting crosslinked hydrogels, may be used include, but are not limited to, facelift procedures, treatment of facial wrinkles, lines, or other facial features.

The tyrHA of the presently disclosed precursor compounds crosslink in the presence of a peroxidase (e.g. horseradish peroxidase) and a peroxide (e.g., hydrogen peroxide) to form a composition comprising (1) a crosslinked tyrHA and (2) an acellular tissue matrix. In certain embodiments, such compositions are in the form of a hydrogel. In certain embodiments, the hydrogel thus formed comprises an aqueous buffer solution. The hydroxyphenyl groups of the tyramine residues of tyrHA react with the peroxide in the presence of the peroxidase to remove the phenolic hydrogen atom, resulting in a tyramine residue free radical, with the unpaired electron associated with the phenolic oxygen atom. This free radical species isomerizes or resonates, resulting in a resonance structure (or free radical isomer) with the unpaired electron now associated with an ortho carbon atom on the phenolic ring. In this position, the unpaired electron quickly reacts with a similarly situated unpaired electron on another tyramine residue free radical to form a covalent bond between them. The result is a free-radical driven dimerization reaction between different tyramine free radical residues attached to different glcAs of the same or different tyrHA molecules. This dimerized species further enolizes to restore the now-linked tyramine residues, resulting in a dityramine linkage structure. It would be understood by persons of ordinary skill in the art that a plurality of reactions as herein described will occur between adjacent tyramine residues, resulting in a crosslinked macromolecular network of tyrHA molecules having the following crosslinking structure of formula (I):

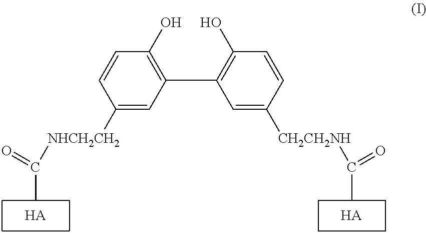

wherein each HA is the same or a different crosslinked tyramine-substituted hyaluronic acid. This crosslinking mechanism is illustrated below in Scheme 2, where R is —CH₂CH₂—.

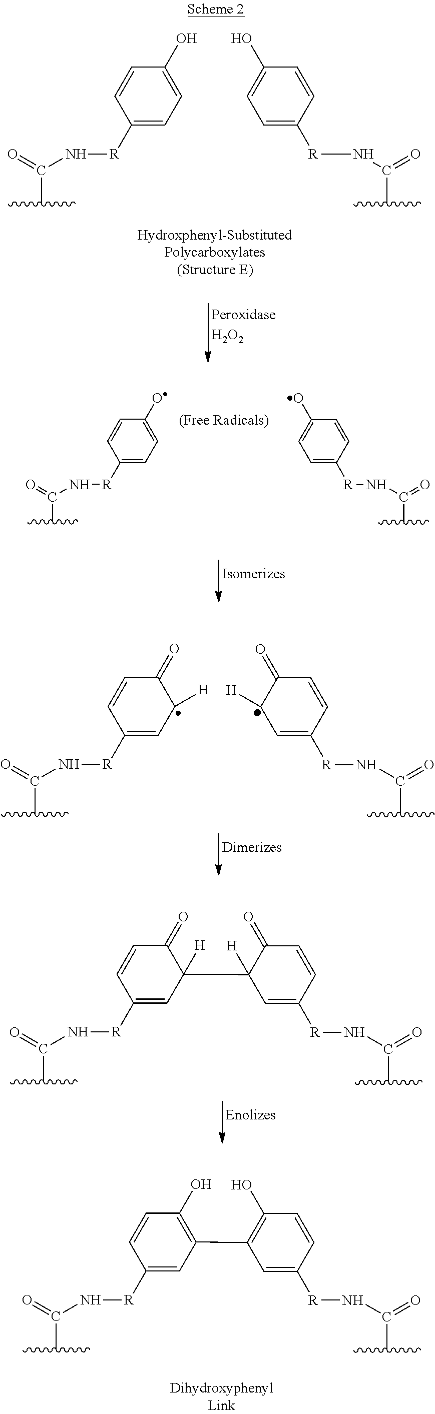

In an alternative, the resulting crosslinked macromolecular network of tyrHA molecules are of the formula (I) or (II):

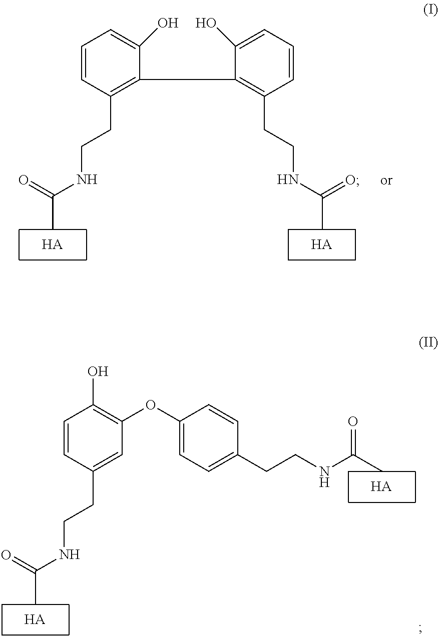

a mixture thereof, wherein each HA is the same or a different crosslinked tyramine-substituted hyaluronic acid. The cross-linking mechanism is illustrated below in Scheme 3. Like Scheme 2, Formula (I) is formed via the reaction of one enol radical with another. In addition, a second reaction takes place between on oxygen radical and the enol radical, therefore forming the product having the Formula (II).

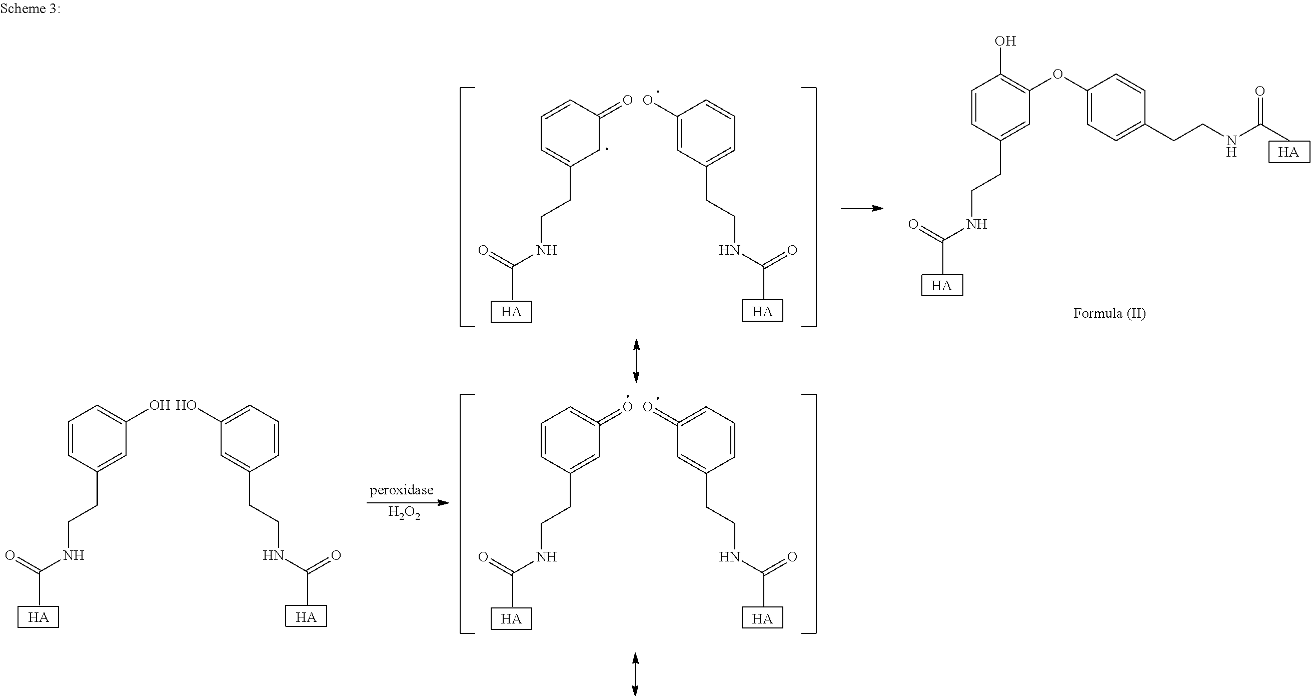

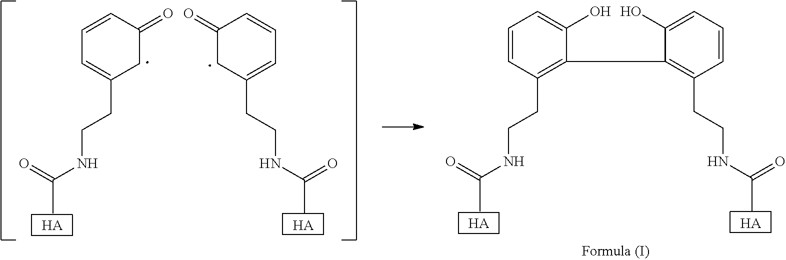

Because the crosslinking reaction is enzyme driven, it can be carried out under ordinary in vivo or metabolic conditions, i.e., temperatures in the range of from 35 to 39° C. and pH in the range of 6 to 7 (e.g. about 6.5). Thus, the crosslinking can be performed in vivo to provide a crosslinked hydrogel at a surgical situs to promote maximum seamless integration between the hydrogel and native tissue. Integration of the hydrogel scaffold with native tissue may occur immediately as the precursor composition quickly penetrates into the existing tissue matrix prior to crosslinking, and crosslinks not only with itself, but potentially with tyrosine residues of resident proteins in the existing tissue matrix. This would mitigate a typical problem found with pre-formed matrix plugs, which is their poor integration into native tissue. The ability to crosslink the hydrogel directly onto the tissue surface eliminates the need to surgically enlarge a defect to fit a pre-cast plug, as is necessary for hydrogels whose chemistries are toxic to or otherwise prohibit their formation inside the patient.

Another aspect of the present invention are kits comprising the presently disclosed precursor compositions. At a minimum, such kits comprise (1) a tyramine-substituted hyaluronic acid and (2) an acellular tissue matrix, as discussed above. In certain embodiments, the kit further comprises (3) a peroxidase, a (4) peroxide, and/or a device capable of mixing components (1), (2), (3), and (4) and/or injecting a mixture of components (1), (2), (3), and (4). In certain embodiments, the device is selected from a group consisting of single barrel syringes, dual barrel syringe systems, cannulae, syringe-to-syringe luer lock adapter-based systems, in-line static mixers, mixing tips, or any combination thereof.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### EXAMPLES

### Example 1—Preparation of a Crosslinked Hydrogel According to the Present Invention

Tyramine-substituted hyaluronic acid (tyrHA) was prepared by dissolving 4% (w/v) hyaluronic acid (HA) in MES buffer and functionalizing it with tyramine (tyr) in a 1:1 molar ratio of tyr to HA carboxyl groups using EDC/NHS coupling. The tyrHA was purified via precipitation or dialysis and then freeze-dried and redissolved in PBS buffer to yield a 25 mg/mL concentration solution. Five U/mL of horseradish peroxidase (HRP) was then added to the tyrHA/PBS solution. Acellular dermal matrix slurry (ADMS) having a 19% solid content was thoroughly mixed with the tyrHA/HRP/PBS solution in a wet weight ratio of 5:1 ADMS:tyrHA (corresponding to a dry weight ratio of 27:1 ADMS:tyrHA) to yield the injectable precursor. The injectable precursor was subsequently contacted with 0.01% hydrogen peroxide (120 μL H₂O₂ per 1 mL tyrHA) to yield a crosslinked hydrogel according to the present invention.

### Example 2—Evaluation of In Vivo Biological Response to the Crosslinked Hydrogel of Example 1

Pucks prepared from the crosslinked hydrogel prepared in Example 1 were subcutaneously implanted into immune competent rats to evaluate certain biological responses (i.e., cell repopulation, revascularization, and inflammation) to these implants after a 4 week time period. It was hypothesized that the implants would retain their shape and integrate with the host tissue by promoting cell infiltration and revascularization in the subcutaneous space.

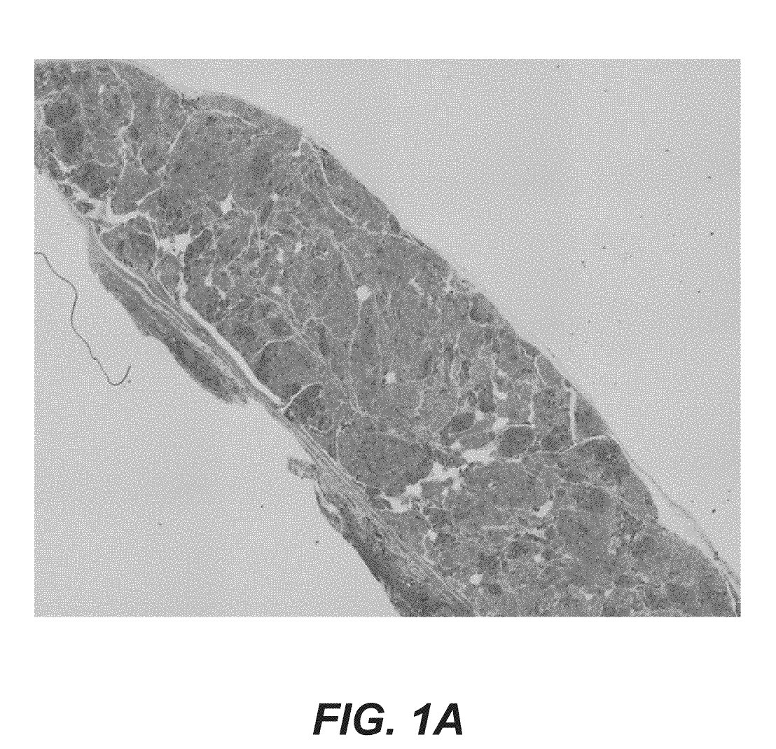

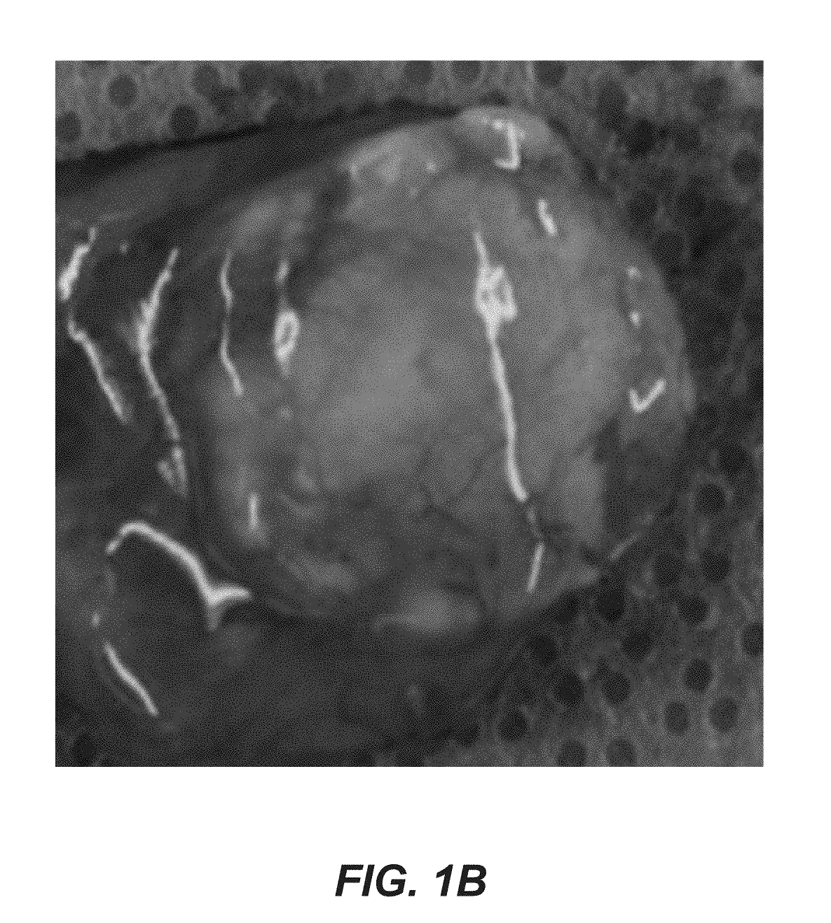

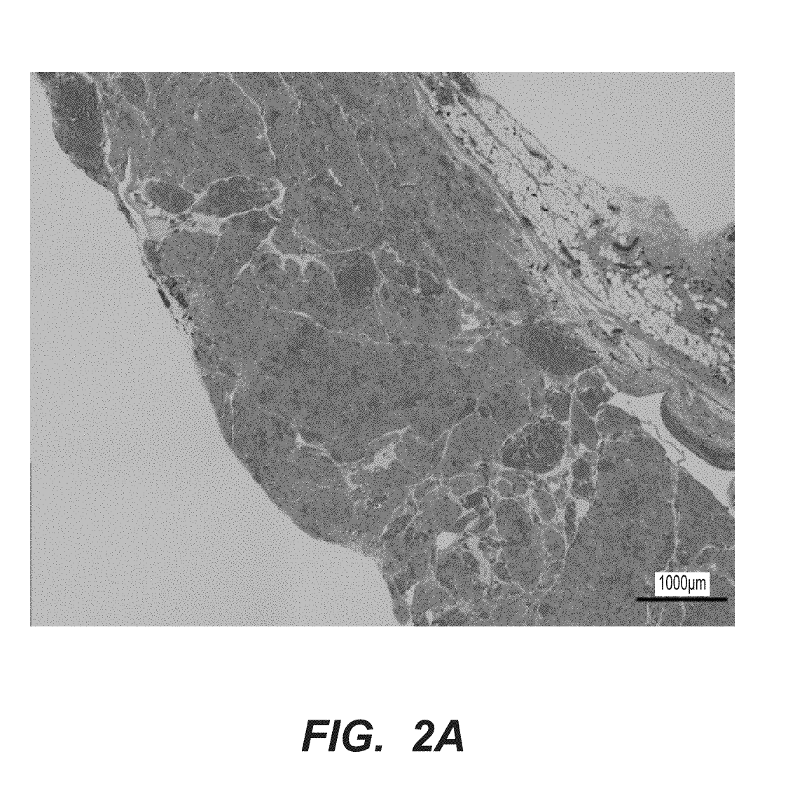

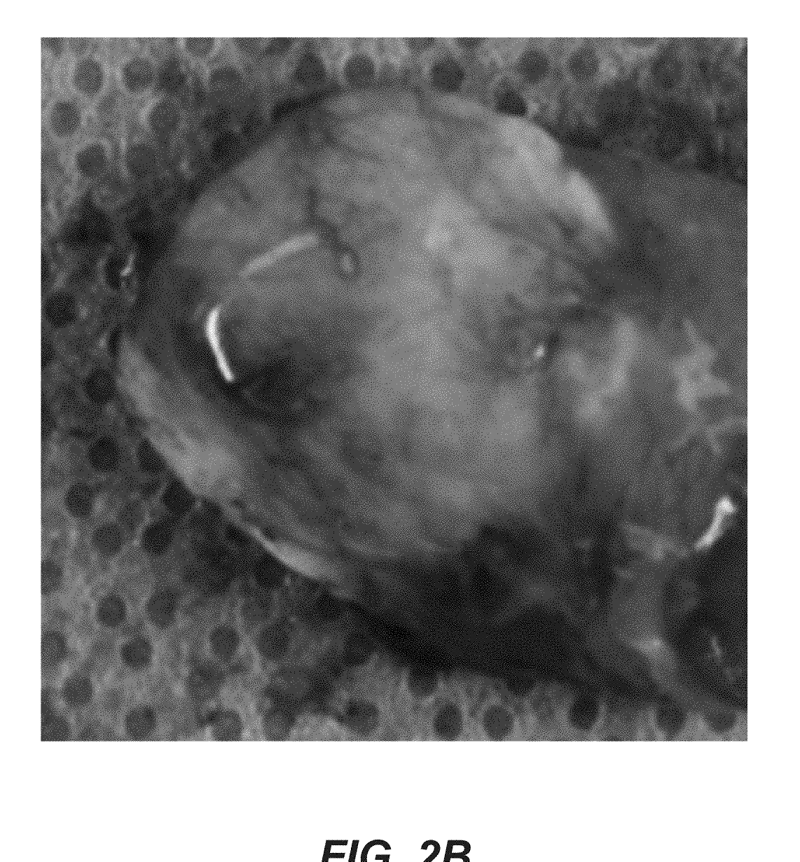

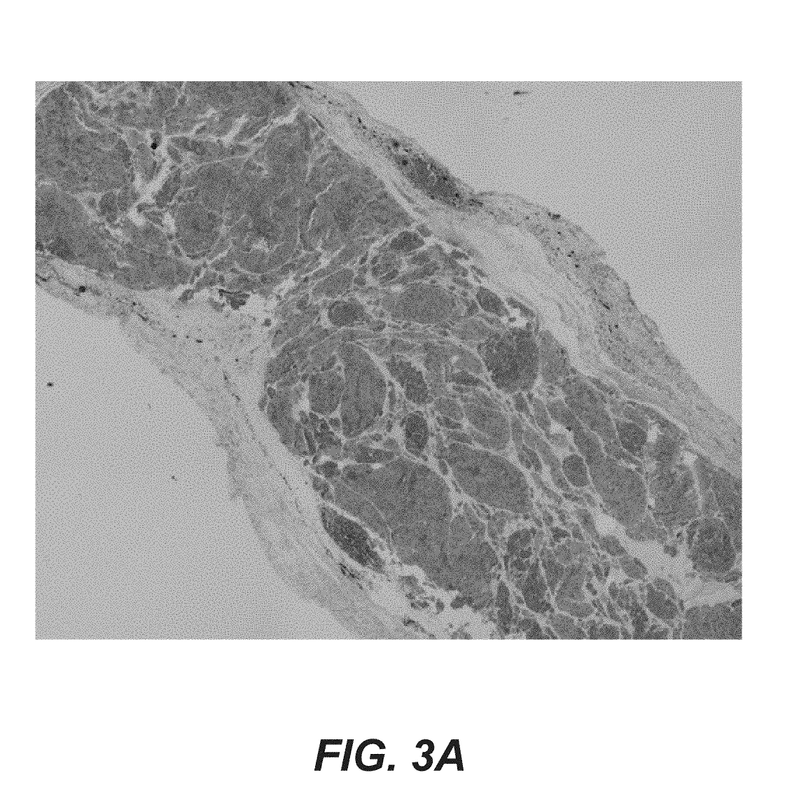

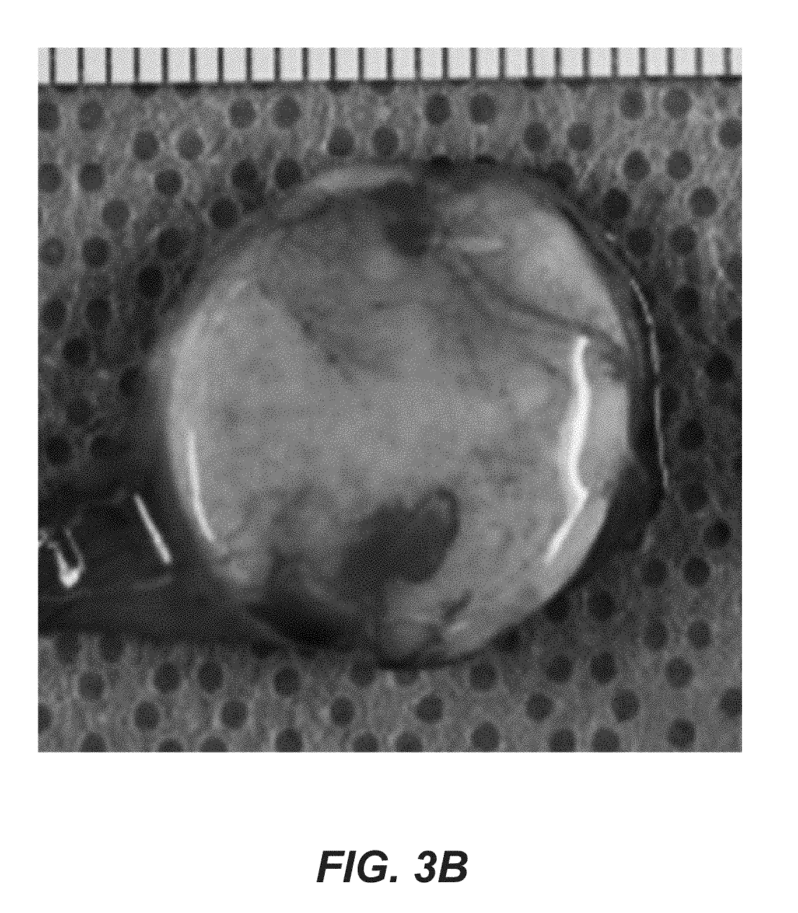

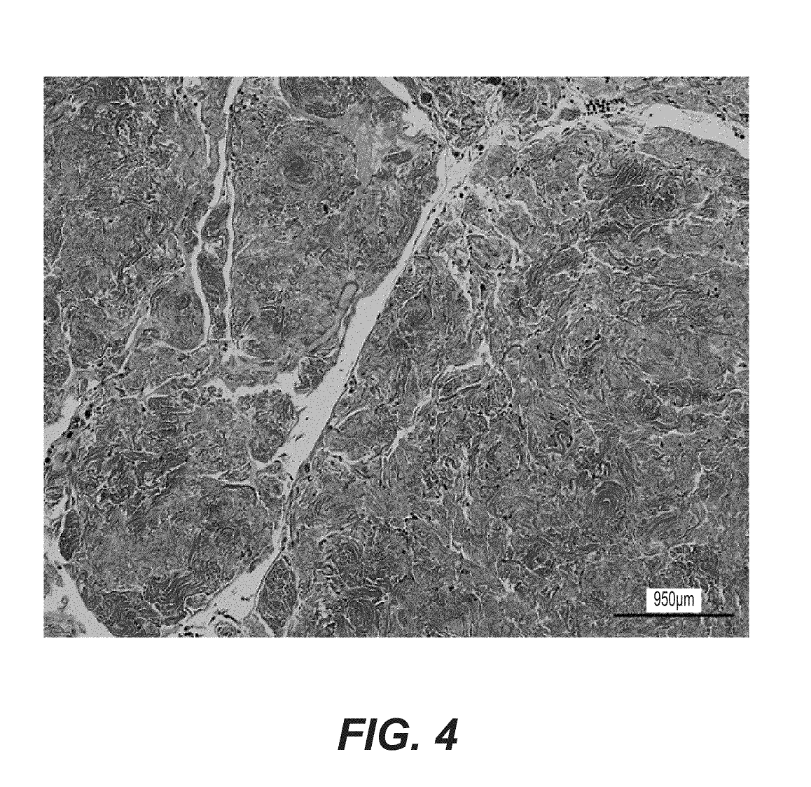

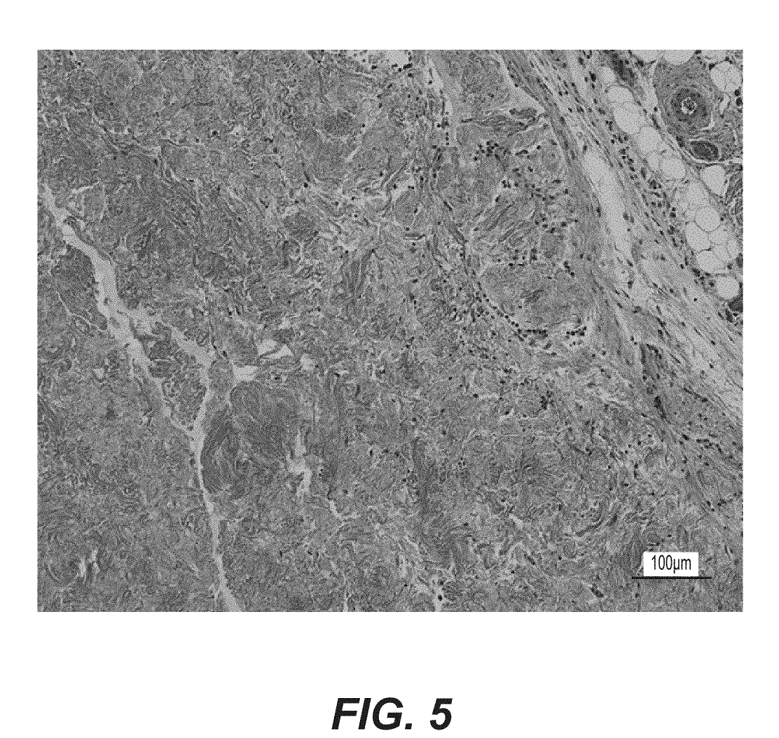

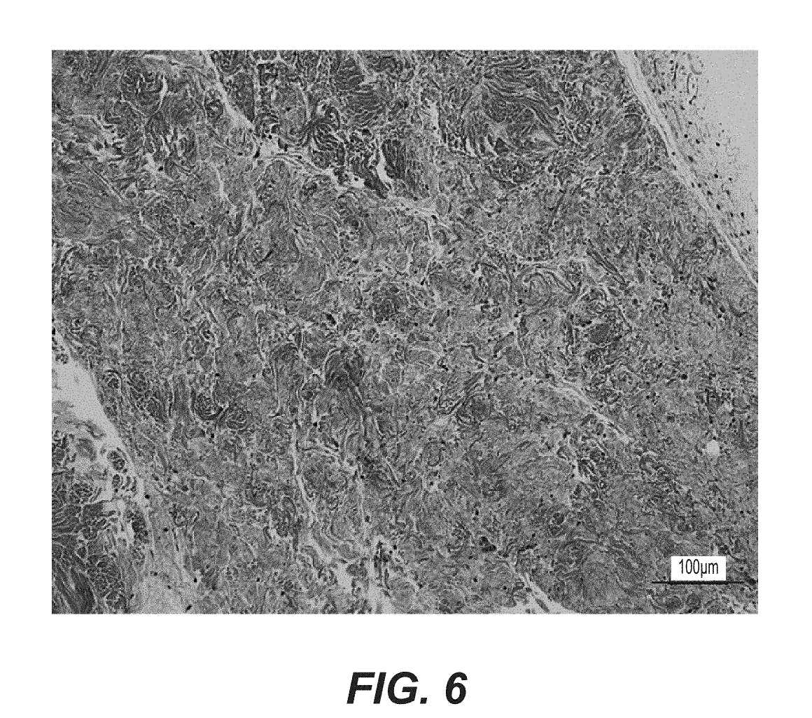

Gross observations of the implants revealed surrounding tissue integration, no signs of inflammation or hematoma, and no dissociation or resorption of slurry. See FIGS. 1b, 2b, and 3b. While the center of the implants remained acellular, H&E histological evaluation of the implant showed a moderate amount of fibroblast-like cell infiltration and revascularization with minimum inflammation. See FIGS. 1a, 2a, 3a, 4, 5, and 6. In contrast, it was observed in the same study that, while no inflammation was observed, ADMS-free, tyrHA-based hydrogel implants prevented cell infiltration, resulting in encapsulation in a fibrotic surrounding tissue layer.

### Example 3—Preparation of a Crosslinked Hydrogel According to the Present Invention

Tyramine-substituted hyaluronic acid (tyrHA) was prepared by dissolving 4% (w/v) hyaluronic acid (HA) in MES buffer and functionalizing it with tyramine (tyr) in a 1:1 molar ratio of tyr to HA carboxyl groups using EDC/NHS coupling. The tyrHA was purified via precipitation or dialysis and then freeze-dried and redissolved in PBS buffer to yield a 25 mg/mL concentration solution. Five U/mL of horseradish peroxidase (HRP) was then added to the tyrHA/PBS solution. Acellular dermal matrix slurry (ADMS) having a 19% solid content was thoroughly mixed with tyrHA/HRP/PBS solution in wet weight ratios of 5:1 and 12:1 ADMS:tyrHA (corresponding to a dry weight ratio of 27:1 and 66:1 ADMS:tyrHA, respectively) to yield two injectable precursors. Each injectable precursor was subsequently mixed with 0.01% hydrogen peroxide (120 μL H₂O₂ per 1 mL tyrHA) to initiate crosslinking using two syringes connected via a luer lock adapter.

### Example 4—Evaluation of In Vivo Biological Response to the Crosslinked Hydrogels of Example 3

Each of the mixtures prepared in Example 3 were subcutaneously injected into immune competent rats and allowed to set into 3-dimensional implants in situ to evaluate certain biological responses (i.e., including fibroblast-like cell infiltration, revascularization, and inflammation) to these implants after a 4 week time period. It was hypothesized that the implants would retain their shape and integrate with the host tissue by promoting cell infiltration and revascularization in the subcutaneous space.

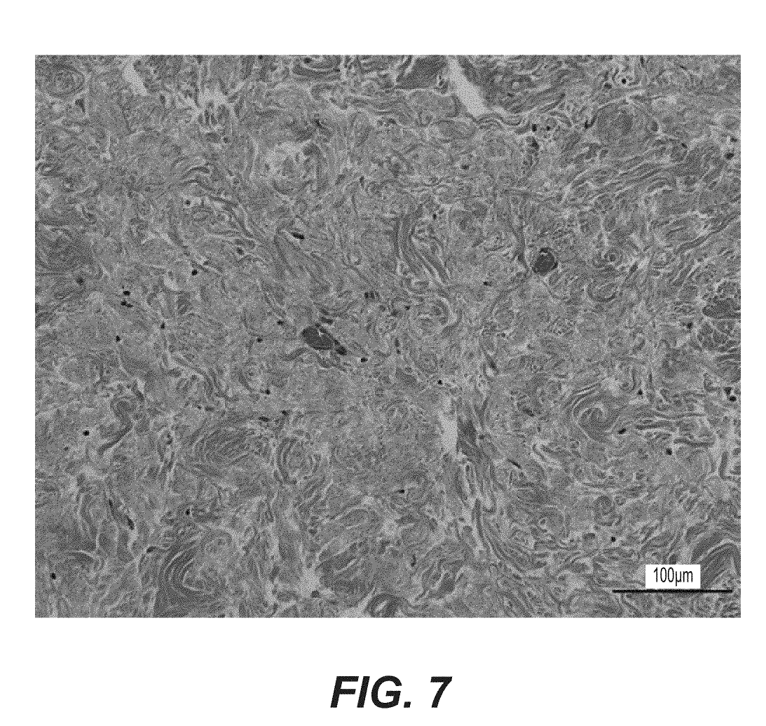

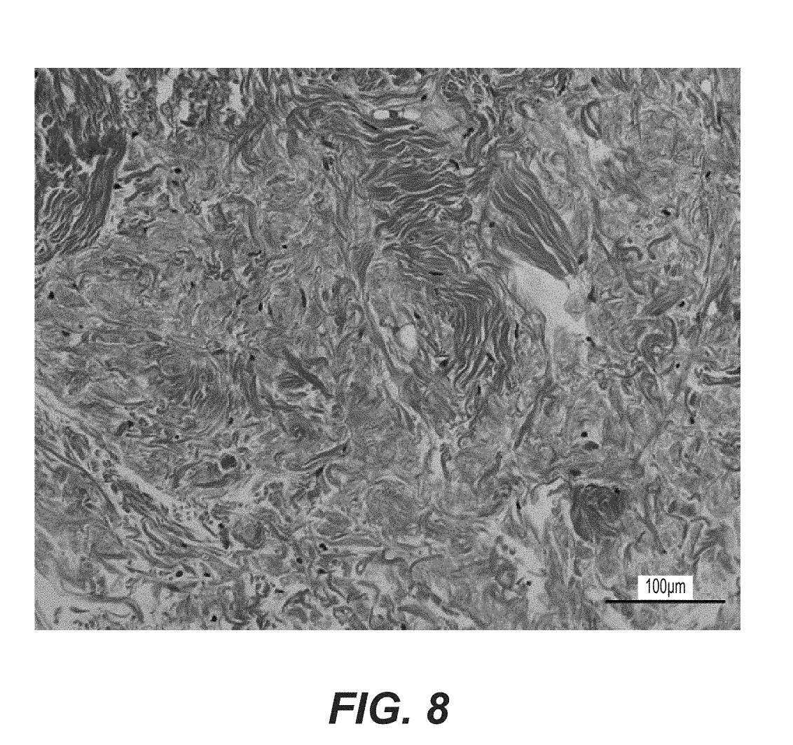

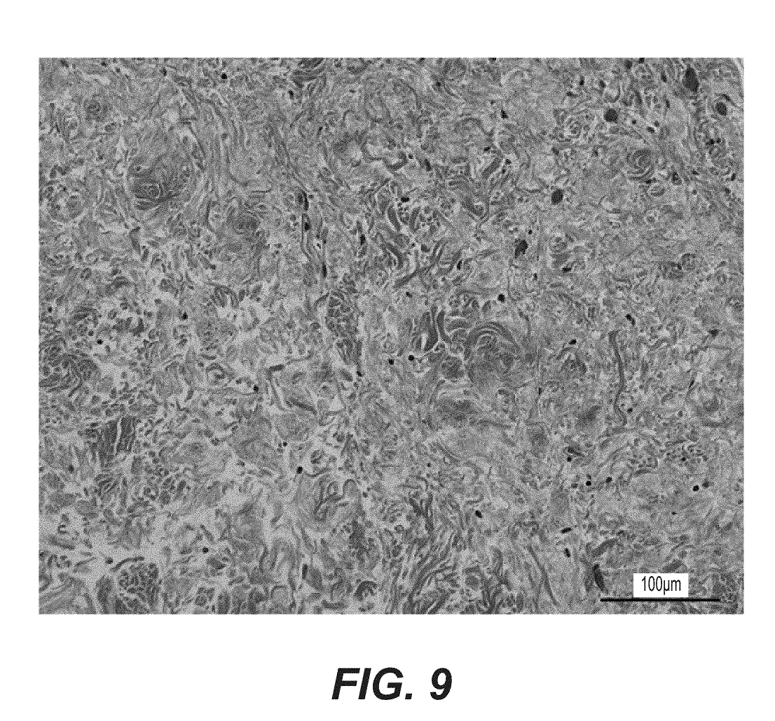

Gross observations of the implants revealed surrounding tissue integration and vasculature, no signs of inflammation or hematoma, and no dissociation or resorption of slurry. H&E histological evaluation of the 12:1 ADMS:tyrHA ratio implant showed a good amount of fibroblast-like cell infiltration and revascularization in the periphery and center of the implants with minimum inflammation, similar to control. While the center of the implants remained acellular, H&E histological evaluation of the 5:1 ADMS:tyrHA ratio implant showed a good amount of fibroblast-like cell infiltration and revascularization in the periphery compared to control (i.e., ADMS alone). See FIGS. 7, 8, and 9. Thus, it appears that increasing the ratio of ADMS to tyrHA in implant leads to faster cell infiltration and revascularization.

1. A composition comprising (1) a tyramine-substituted hyaluronic acid and (2) an acellular tissue matrix.

2. The composition of claim 1, wherein the composition further comprises a peroxidase.

3. The composition of claim 2, wherein the peroxidase is horseradish peroxidase.

4. The composition of claim 1, wherein the tyramine-substituted hyaluronic acid is derived from a hyaluronic acid having a molecular weight in the range of about 1.5 MDa to about 1.8 MDa.

5. The composition of claim 1, wherein the tyramine-substituted hyaluronic acid is derived from a hyaluronic acid selected from a group consisting of human-derived hyaluronic acid, porcine-derived hyaluronic acid, bovine-derived hyaluronic acid, bacteria recombinant hyaluronic acid, rooster comb hyaluronic acid, or any combination thereof.

6. The composition of claim 1, wherein the acellular tissue matrix is derived from dermal tissue, adipose tissue, muscle tissue, bone tissue, cartilage tissue, or any combination thereof.

7. The composition of claim 1, wherein the acellular tissue matrix used to form the composition is in the form of a slurry, diced tissue particles, a cryomilled dry powder, micronized dry particles, or freeze dried porous sponge particles.

8. The composition of claim 1, wherein the tyramine-substituted hyaluronic acid is present in the composition in a concentration of up to 25 mg/mL, based on the total volume of the composition.

9. The composition of claim 1, wherein the units of activity per volume of the horseradish peroxidase in the composition is in the range of from 0.5 U/mL to 50 U/mL, based on the total volume of the composition.

10. The composition of claim 1, wherein the dry weight ratio of tyramine-substituted hyaluronic acid to acellular tissue matrix in the composition is in the range of from 1.0:1.0 to 1.0:100.0.

11. The composition of claim 2, wherein the composition is in the form of a liquid.

12. The composition of claim 11, wherein the composition comprises water.

13. The composition of claim 12, wherein the composition comprises an aqueous buffer solution or a preservation solution.

14. The composition of claim 11, wherein the composition is in the form of a solution, a suspension, a dispersion, or any combination thereof.

15. The composition of claim 1, wherein the acellular tissue matrix has been sterilized.

16. The composition of claim 15, wherein the acellular tissue matrix has been sterilized via e-beam, gamma radiation, UV radiation, and/or supercritical CO₂.

17-36. (canceled)

37. A method of filling a void in a tissue of a human or animal comprising:
(a) providing an aqueous composition comprising (1) a tyramine-substituted hyaluronic acid, (2) an acellular tissue matrix, and (3) a peroxidase;
(b) providing an aqueous solution of hydrogen peroxide;
(c) mixing the aqueous composition of (a) and the aqueous solution of (b) to form a mixture and initiate crosslinking of the tyramine-substituted hyaluronic acid;
(d) introducing the mixture of (c) into the void in the tissue of the person or animal to be filled such that the void is filled and the crosslinking of the tyramine-substituted hyaluronic acid is completed in situ.

38. The method of claim 37, wherein the peroxidase is horseradish peroxidase.

39. The method of claim 38, wherein the acellular tissue matrix is derived from dermal tissue.

40. The method of claim 37, wherein the void is an anal fistula in a human.

41. The method of claim 37, wherein the acellular tissue matrix has been sterilized prior to step (a).

42. The method of claim 41, wherein the acellular tissue matrix has been sterilized via e-beam, gamma radiation, UV radiation, and/or supercritical CO₂.

43-47. (canceled)
